(19) United States
(12) Patent Application Publication
(10) Pub. No.: US 2003/0223947 A1
(43) Pub. Date: Dec. 4, 2003

(54) Multi-phased immiscible liquid composition for toy articles

(75) Inventor: Gilda C. Alvarez, Redondo Beach, CA (US)

Correspondence Address: Robert W. Mulcahy, 520 Sequoia Drive, Sunnyvale, CA, 94086, US

(73) Assignee: Mattel, Inc.

(21) Appl. No.: 10/440,772

(22) Filed: May 19, 2003

Related U.S. Application Data
(60) Provisional application No. 60/384,765, filed on May 31, 2002.

(51) Int. Cl. A61K 7/06; A61K 7/11; C09K 19/52; C09K 19/02

(52) U.S. Cl. 424/70.12; 252/299.01; 349/182

(57) ABSTRACT

The invention relates to the field of optical toys and amusement devices. More specifically, the invention is directed to specific immiscible liquid and semi-liquid compositions of different specific gravities and viscosities and their placement in enclosed spaces or chambers of various toy or entertainment devices thereby creating a multi phase liquid system having enhanced or dynamic visual effect. The invention employs a novel immiscible liquid mixture of hydrophobic alkane hydrocarbons and polar organic esters. In operation, as the two phase liquid system is agitated or otherwise energy stressed, the liquid of the heavier specific gravity flows through the liquid of the lighter specific gravity in distinct spaced apart beads or droplets under the influence of gravity.

This application claims benefit of U.S. Provisional Application No. 60/384,765, filed May 31, 2002, which is hereby incorporated by reference in its entirety.

### BACKGROUND OF THE INVENTION

1. Field of the Invention

The present invention relates to the field of optical toys and amusement devices and compositions for same. More specifically, this invention is directed to compositions of a plurality of mutually immiscible liquids and semi-liquid gels which generate visual effects in display devices or as applied gel compositions. The liquid compositions may be used in visual effect articles employing sealed chambers, tubes or other sealed arrangements wherein the movement of a liquid of one specific gravity with respect to another immiscible liquid of a second specific gravity generates eye-catching visual effects. Alternatively the immiscible liquids may be combined with a thermoplastic gel to form a visually attractive cosmetic composition.

2. Description of the Related Art

This invention relates to toy and entertainment display compositions and devices and in particular to a visual effect device utilizing a sealed arrangement for enclosed liquids and the movement of a liquid of one specific gravity with respect to a liquid of a second specific gravity within the enclosure. The present invention belongs to a category of entertainment compositions and devices which utilize visual effects produced when two or more contrastingly colored liquids, immiscible in one another, are placed in a container and subjected to physical action such as heating, shaking, change of orientation and the like. More specifically, this invention is directed to visual devices which consists of one or more sealed liquid containers in which at least two immiscible liquids are constrained to flow in spatially confined volumes and in controlled directions so as to cause varying visual designs as the two immiscible liquids move relative to one another. Alternatively, these unique immiscible liquid compositions can be mixed in a gel matrix to form a high shine semi-liquid gel cosmetic composition.

One example of such visual devices employing immiscible liquid systems is known under the commerical name “Lava Lamp”, in which two contrastingly colored, immiscible liquids are contained in a bottle-like chamber with a heat source located in the base. The heat from the source causes one of the two liquids to in an effect “boil,” producing a slow but observable upward flow of large globules of the one liquid relative to the other. Upon moving away from the heat source, the globules cool and tend to settle toward the heat source. Thermal currents in the liquids produce an ever changing geometry of the globules. Light directed through the bottle-like chamber enhances the visual effect produced. When the globules settle they are again reheated and the process repeats itself producing a continuously changing display until the heat is removed. As applied to the instant invention, specific immiscible liquids of different specific gravities and selected viscosities are placed in sealed chambers or tubes of various toy devices thereby creating a two phase liquid system therein which when agitated causes a globular interaction resulting in a “lava lamp” visual effect. In operation, as the two phase liquid system is agitated or otherwise energy stressed, the liquid of the heavier specific gravity flows through the liquid of the lighter specific gravity in distinct spaced apart beads or droplets under the influence of gravity. The series of obstructions or various shapes and geometric configurations created by the displacement in the two phase system create a visual display in the toy.

Mutually immiscible liquids are those which after intimate mixing with every other liquid phase of the system maintain separate liquid phases at equilibrium. No matter how thoroughly the liquids are mixed, they will always separate into the same number of layers on standing. Other devices utilize the influence of gravity to produce visual effects. One such device is of the type described in Calfee U.S. Pat. No. 3,564,740 in which a multi-compartment display device is provided with liquids of different specific gravities stored therein. The visual effect produced by the relative movement of the liquids is obtained by inverting the device like an hour glass to produce the gravity flow of the heavier liquid in the downward direction with the consequent upward displacement of a lighter liquid. The flow of liquids is accomplished by a valving or porting arrangement in barriers or partitions which divide the several compartments of the display device, one from the other.

The present invention is directed to a novel two phase immiscible composition usable in toy devices and simple in its admixture thereby eliminating the use of special ports or valves (single or in symmetrical pairs) and utilizes a basic chamber reservoir for containment and operation. The present invention is a substantial improvement over the prior art “lava lamp” compositions and devices providing liquid combinations utilizing liquids other than plain water and heavy oils to produce the droplet or beading effect characteristic of the present invention without the need of special ports or valves in the toy devices that house the instant compositions.

Regardless of the type of liquids used in toy articles, it has remained imperative that such materials be safe for young children as well as practical for making a relatively inexpensive consumer device. Safety requirements have evolved through the years as safety concerns have grown to the point that any such liquids should be inexpensive, non toxic, non-combustible, easy to dye, and liquid throughout the range of normal room temperatures and pressures. More specifically, safety requirements mandate that any material compositions used in a toy be odorless, nonirritating to the skin or eyes or the like, and be nontoxic if ingested. Accordingly, such liquid compositions must avoid the use of undesirable volatile organic liquids the residual presence of which can be dangerous due to the toxic effect of certain aromatic and chlorinated hydrocarbon liquids. And even further additional requirements have been expected of these materials to generally avoid damage to clothing, upholstery fabric, or carpeting even though such materials may be encased in a toy. What is needed are room temperature, mutually immiscible liquid systems of inexpensive, non-toxic, non-combustible, readily dyeable organic compounds compatible with optical plastics and thermoplastic gels.

In view of the environmental, health and safety concerns in the use of highly toxic organic liquids, less controversial liquids such as water or mineral oil have been used to provide a liquid media in toy devices. However, water and mineral oil have their own disadvantages. The present invention is directed to a multi-phased novelty fluid system for use in toy and entertainment devices and compositions to create a visual effect in these devices and compositions. The fluids in the present system are: (a) readily available, (b) proven safe for use in toy products, (c) of moderate cost, and (d) produce a novel multi-phased immiscible liquid composition which (1) when stressed to cause admixture in a sealed system creates a distinct flow pattern resulting in a unique “lava lamp” effect visual display and (2) when added to a thermoplastic gel engenders a semi liquid composition which when applied as a skin cosmetic forms a high shine colorful layer.

### SUMMARY OF THE INVENTION

The present invention provides novel compositions which make possible a unique combination of mutually immiscible liquid phases than have been practical in entertainment and toy devices up to now. It provides novel systems of two, three, four, and five mutually immiscible liquids, and it provides systems of liquids which are non-toxic and non-combustible, do not attack commonly used molded plastics such as poly(vinyl chloride), polycarbonate, nitrile, and acrylic resins, and are relatively easy to dye to different pure colors. It makes possible improved visual display devices and compositions. One of the principal uses contemplated for this invention is in visual toys, especially in novel toys wherein mutually immiscible liquid phases are enclosed in a sealed liquid container, e.g. between two substantially parallel, transparent, flexible sheets of transparent thermoplastic resin or encased in a sealed channel flow arrangement.

While systems of two or more liquid or semi-liquid phases are not unusual, they are rarely encountered in ordinary everyday experience and tend to be curiousities as in the case of multiphase liquid “lava lamps” or gasoline slicks on water. Indeed, the normal expectation is that whenever a number of different organic and aqueous liquids are mixed, there will be the usual two phases, an oil phase and a water phase. While achieving multiple phases is common, achieving mutually immiscible liquid phases using inexpensive non-toxic, non-combustible, easily dyeable, and readily available substances is substantially more difficult. The multiphased immiscible liquids of the instant invention have these necessary characteristics and are acceptable as liquids for use in consumer toy and entertainment products.

In accordance with the present invention, there is provided a multi-phased liquid composition comprised of immiscible liquids of different specific gravities and viscosities, one phase significantly heavier than any other. The discovery of the present invention is that one may formulate a superior multiphased visually active type composition by combining a first phase liquid comprised of one highly hydrophobic substance with a second liquid phase containing polar organic compositions of certain organic esters. The hydrophobic liquids of the multiphased compositions herein are selected from the groups of (1) aliphatic alkanes hydrocarbons having in excess of six carbon atoms, (2) silicone oils and (3) low molecular weight organic polymeric liquids. The polar organic liquids contemplated herein are the certain saturated alkyl organic esters of carboxylic or fatty (C3-C₁₈) acids wherein the alkyl groups of the ester can contain from 3 to 30 carbon atoms (C₃-C₃₀). Examples of preferred hydrophobic alkanes used in the instant multiphased immiscible liquid system include alkane hydrocarbons of up to 20 carbon atoms such as isododecene, isohexadecane, and isoeicosane. Exemplary hydrophobic silicone oils include dimethicone and cyclomethicone. Typical low molecular weight organic polymer liquids include polyolefins such as low molecular weight liquid polybutene and low molecular weight co-polymers of ethylene, propylene, and butylene.

The heart of the instant invention lies in the discovery of special polar organic compounds in the form of esters which when used in combination with certain hydrophobic liquids form multiphased immiscible liquid compositions having the unique quality of usage set forth herein. These esters are those carboxylic acid ester derivatives selected from the family of long chain alkyl carboxylic acids (fatty acids) wherein the alkyl group (of the acid portion) contains at least seven carbon atoms. A preferred range of carboxylic acids contains an alkyl group of from C₇ to C₂₀ and include valeric, caproic, caprylic, capric, lauric, myristic, palmitic, stearic, and oleic acids. The alcohol portion of the esters are organic alkyl alcohols containing up to 8 carbon atoms and includes ethyl alcohol, n and iso-propyl alcohol, n and isomeric butyl alchohol, n and isomeric pentyl alcohol, n and isomeric hexyl alcohol, n and isomeric heptyl alcohol, n and isomeric octyl alcohol and include isopropyl, trimethyl butyl, and 2-methyl propyl alcohol. Some preferred organic esters of the instant compositions include isopropyl myristate or isopropyl palmitate, Triisodecyl trimellitate can also be used in this invention.

All of the multiphased (two phases or more) immiscible liquids of the instant invention are particularly useful in visual display devices where each of the liquid phases of the composition are colored, enclosed in a sealed container or channel, and mixed to effect a mutiphased liquid visual dynamic system that is vibrant and pleasing. In another embodiment of the instant invention the multiphased immiscible system can be combined with a semiliquid gel such as a hydrogenated butylenes/ethylene/styrene copolymer or a heavy liquid such as glycerin, mineral oil, or propylene glycol to form a multiphased, glossy, cosmetic in the form of a lip gloss for use by children.

In yet another variation, water may be substituted or added as a component of the immiscible multiphased system. These immiscible liquids of differing specific gravity and viscosity form a multi-phased immiscible liquid mixture of completely unexpected character in that it possesses exceptional mixed rheology resulting in a free flowing liquid mixture which when subjected to stress and intermixture creates a distinctive flow pattern in the nature of a “lava lamp” or highlighted visual effect. The nature of the instant composition enables its use in a host of play patterns or toys for children or adults which exploit its free flowing intermixture and dynamic visual effects.

In yet another distinct variation of the instant visually active immiscible liquid mixture compositions there is further disclosed and provided herein a two phased composition comprised of (I) a first heavier (higher specific gravity) liquid composition comprised of:

(i) a salt such as sodium chloride;

(ii) ethylenediamine tetra-acetic acid (EDTA) and/or propylene glycol (or water); and

(iii) a blend of polyacrylamide,C13-14 isoparaffin, and laureth-7 (a surfactant);

and (II) a lighter liquid (lower specific gravity) phase comprised of a heavy oil such as a polyalpholefin oil of the type described herein and preferably comprised of 1-Decene, hydrogenated homopolymer. This immiscible liquid mixture embodiment can be used as an additional admixture to the hydrophobic/polar ester immiscible mixtures described above.

When a two phased system is mixed, the heavier liquid flows through the lighter liquid creating a display of distinct flow patterns in a “lava lamp” visual effect. The specific gravity of the heavier phase is generally above 1 while the lighter phase is less than 1. The instant multiphased liquid composition is generally place in a transparent or translucent containment vessel. In the use of a two phased system, when the two liquid phases are stressed (by heat or physical admixture), the heavier liquid intermixes with the lighter liquid forming a beaded or globular pattern of heavier liquid throughout the lighter phase in a “lava lamp” effect. The vessel generally has an illumination source which when activated highlights the distinctive “lava flow” of the two liquids and generating a dynamic visual effect.

Specifically provided herein is an entertainment or toy display device which depends on the movement of a plurality of mutually immiscible liquids and contains a mixture of at least two mutually immiscible liquids, one of which is a hydrophobic liquid comprised of a composition selected from the group comprising (a) an alkane hydrocarbon liquid, (b) a silicone oil, and a low molecular weight polyalphaolefin, and at least one other is a polar fatty acid ester liquid. Alternatively provided is the instant multi-phase immiscible composition in a polymeric gel or semi-liquid to form a multiphase lip gloss composition.

### DETAILED DESCRIPTION OF THE INVENTION

The present entertainment device employing the instant novel multiphased liquid compositions is the combination of three key features: (1) the use of a widespread available visual display immiscible liquid materials; (2) the use of widely available and toy (safety) tested liquid compositions; (3) the employment of particular polar organic compositions in the form of fatty acid esters; and (4) the heretofore unknown use of polyalphaolefin oils as a hydrophobic phase in “lava lamp” visual effect compositions. Of course other equivalent materials may be used, such as mineral oil which, while not preferred and, in fact, is discouraged, can be used in place of the alkane liquid hydrocarbon, the silicone oils, or the polyalphaolefin oil of the instant multiphased liquid system or as additional phase materials. And further, as indicated, glycerin or propylene glycol can additionally be used in the instant multi-phased liquid compositions.

The invention basically comprises the instant multiple phase immiscible liquid compositions encased in a toy or entertainment device container comprising a translucent or transparent housing for a main chamber reservoir in the form of a sealed vessel or a tubular channel arrangement all of which is encased by the housing. The device is manipulated under the force of gravity or physically actuated so as to cause the flow of each phase over that of the other to effect a dynamic visual display of the multiphased liquids of varying specific gravity. As can be visualized energy applied to intermix two phases herein causes formation of beads of liquid ascending or descending from one liquid phase to another as it encounters the obstruction and flow of the other phase under the influence of gravity or other force and in the direction controlled by the obstruction.

The invention is specifically directed to a multiphased visually active immiscible liquid composition by combining (A) a first phase liquid comprised of one highly hydrophobic liquid composition and (B) a second liquid phase containing polar organic compositions of certain organic esters, each composition being immiscible with the other. As indicated, the hydrophobic liquids herein are selected from the groups of (1) alkanes having in excess of six carbon atoms, (2) silicone oils and (3) low molecular weight organic polymeric liquids. The polar organic liquids contemplated herein are the certain alkyl organic esters of carboxylic acids wherein the alkyl groups of the ester can contain from 7 to 30 carbon atoms (C₇-C₃₀). Other liquids immiscible with the two basic group listed may be added to form multiphased (more than two) immiscible liquid mixtures. These multiphased liquid compositions are used in entertainment or toy devices for purposes of dynamic visual display of the flow characteristics of multiphased liquids.

The hydrophobic alkanes used in the hydrophobic phase (A) of the instant multiphased immiscible liquid system include alkane hydrocarbons of up to 20 carbon atoms such as isododecene, isohexadecane, and isoeicosane. Heavier alkane liquids having in excess of 8 carbon atoms (C₂₀) can also be employed herein. These alkanes have been found to be readily available, safe for use in children's toys, and non-flammable. Other alkane hydrophobic liquids usable herein include nonane, decane, undecane, tetradecane, hexadecane, octadecane, and isomers thereof.

The hydrophobic silicone oils usable in the present invention include dimethicone and cyclomethicone. Various other silicones can be employed in the hydrophobic phase (A) such as Dow Corning 200 Fluid, a dimethyl polysiloxane, both grade 100,000 CS viscosity and grade 600,000 CS viscosity. Still other commercially available silicone liquids can be used as hydrophobic phase (A).

Typical low molecular weight organic polymer liquids include polyolefins such as low molecular weight liquid polybutene and low molecular weight co-polymers of ethylene, propylene, and butylene. Other suitable polyolfin liquids that can be operable and functional in hydrophobic phase A herein include hydrogenated butylenes/ethylene/styrene copolymer.

The polar organic esters herein are selected from a preferred range of carboxylic (fatty) acids containing an alkyl group having carbon content of from C₇ to C₂₀ and includes valeric, caproic, caprylic, capric, lauric, myristic, palmitic, stearic, and oleic acids. The alcohol portion of the esters are organic alkyl alcohols containing up to 10 carbon atoms and include ethyl alcohol, n and iso-propyl alcohol, n and isomeric butyl alchohol, n and isomeric pentyl alcohol, n and isomeric hexyl alcohol, n and isomeric heptyl alcohol, n and isomeric octyl alcohol and include isopropyl, trimethyl butyl, and 2-methyl propyl alcohol. Preferred esters are alkyl palmitates and myristates and include isopropyl myristate and isopropyl palmitate.

Again, the present multiphase immiscible liquid compositions are uniquely useful in display and visual activity products. Display devices made of poly(methyl methacrylate) or poly(vinyl chloride) containing two immiscible liquids are currently being sold commercially. The present invention makes possible two-, three- and four-phase immiscible liquid systems which can be used in those same devices and are not toxic, not combustible, and relatively inexpensive and nonvolatile.

If a liquid layer formed according to the principles above is not inherently colored, a dye or other coloring matter should preferably be added to form a colored liquid layer. Preferably, coloring agents should be chosen so that each layer is a different color and the combination of all layers taken together is aesthetically agreeable. Pleasing combinations can also be created when some layers are colored and others are not. In addition to dyes, small quantities of other materials may be added to achieve various purposes, such as stabilizing or destabilizing emulsions, increasing or decreasing viscosity of density, improving component stability, or modifying solubility properties to control the ratio of concentration of dye in two layers. Minor amounts of various materials may be present as impurities. When a phase is said to be “composed primarily” or “consisting essentially of” of a class of substances, it should be understood that minor amounts of other materials may also be present, and that all materials which are major constituents of any of the other liquid phases will be minor constituents of every phase in equilibrium with them. In all cases herein, however, a phase composed primarily of something must contain a minimum of 50% by weight of that material.

Preferably, all liquids used herein are transparent so that lines, shapes, bubbles, and colors may be seen through them. If the liquids are translucent or opaque, some of the unique visual effects created by overlapping, interpenetrating, or suspended liquids become obscured or invisible, as do other effects depending on light shining through the liquids. The visual effects observed depend upon the geometry and transparency of the container and the color, viscosity, density, transparency, index of refraction, and surface tension of the liquids. Preferably, the densities of the liquids are sufficiently different that emulsions separate by gravity reasonably rapidly.

This invention contemplates use of liquid compositions in entertainment and toy display devices which depend on movement of a plurality of mutually immiscible liquids. Often the movement is vigorous, as in most of the prior art examples cited above, eg. “Lava Lamps”. It can also be very slow. Sometimes the devices are designed to sit around as decorative objects in which most of the time no motion is apparent. In all cases, however, the devices depend in part for their effect upon the fact that under certain conditions the liquids can be observed to move. Liquids which have relatively low viscosity and therefore move relatively rapidly are preferred.

The compositions of multiple, mutually immiscible liquid phases of this invention can be combined whenever desired with gas or solid phases, or with additional immiscible liquid phases such as mercury. They can be confined in any sort of container or apparatus with which they are chemically compatible, including containers described above and those available commercially or otherwise in the prior art.

One embodiment of this invention is a display device wherein a typical recipe of the instant composition of colored liquids is confined within a container the principal walls of which are two substantially parallel, transparent sheets. When this device is moved so as to induce movement in the liquids, such as for instance by tilting it, interesting and amusing shapes, colors, and patterns can be obtained. Another embodiment is in a toy where the instant immiscible liquids are confined in a sealed translucent extended pipe arrangement such as in a small action toy where the liquid is driven through the tubular structure thereby generating the colorful patterns. Examples of different display devices embodying liquid systems of the present invention are profusely available in the prior art.

With a flexible device, patterns and movements of color can be created and moved around in a way that must be seen to be appreciated. Multiple colors overlap, change, blend, and flow. Many of these effects cannot be created merely by gravity flow, or by shaking a rigid device, nor could the magnitude of their aesthetic impact be anticipated by knowing that liquids confined behind flexible walls can be made to move. As the changing colors stream and drift, coalesce and divide, emulsify and regroup, visual effects are created which are not obtainable with known devices. For example, by picking up the device and exerting different amounts of pressure in various locations using ones fingers, one can force the walls together at any of a wide range of variable locations and in that way create a temporary channel separating but connecting two reservoirs of colored liquids; and then by adjusting the finger forces on the container one can cause blobs of colored liquids to stream through the channel, maintaining their own original color but elongating their shapes in a rapidly flowing multicolored river flowing from one reservoir to the other. When all the finger forces are removed, the container will preferably spontaneously revert to its original sheet-like form.

In another embodiment of the instant invention, a multiphase cosmetic composition in the form of a lip gloss is provided herein by using the immiscible liquid systems of the present invention. Specifically, a two phase lip gloss is prepared by mixing the instant hydrophobic phase (A) and polar organic phase (B) immiscible mixture in a gel composition comprising a relatively low molecular weight alpha olefin copolymer (MW 1,000-25,000) and glycerin. In employing the instant hydrophobic phase/polar organic phase immiscible liquid mixture, an extremely stable lip gloss system is provided by a glycerin gel system in combination with an polyolefin copolymer, preferably hydrogenated butylene/ethylene/styrene copolymer. The presence of the polyolefin copolymer in the proper proportions retards viscosity development and maintains the resulting gel at a constant stability over a broad range of temperatures and over a long period of storage. The instant multiphase lip-gloss is primarily useful as a child's toy cosmetic due to the safe attributes of all ingredients.

In preparing the polyolefin copolymer-glycerin gel systems usable in the present invention, it is noted that polyolefins having molecular weights between 1,000 and 25,000 may be used. Polyolefins with molecular weights below this level provide weak, unstable gels, while those of higher molecular weight, require too long a time to dissolve, with a resulting excessive heating of the mineral oil constituent. A preferred polyolefin copolymer is hydrogenated butylenes/ethylene/styrene copolymer.

In accordance with another preferred embodiment of the present lip gloss invention, a wear-enhancing agent such as a poly iso and normal butene, lanolin, or dimethyl polysiloxane is added. This material makes the lip gloss more waterproof and permits it to retain its coloring and other beneficial effect on the wearer's lips for a substantially longer period. Without the agent, the lip gloss disappears within about 30 minutes. The level of wear-enhancing agent used may vary, depending on the extent of improvement desired. Where the wear-enhancing agent is poly iso and normal butene at a level of about 43% of the total composition, lip gloss has been found to last as long as three hours on the lips of the wearer. Lesser amounts provide reduced wear. Amounts substantially in excess of this amount have adverse affects on the stability of the gel.

Dyes which have been found suitable for use in the instant two phased novelty composition and entertainment device are ordinary food coloring dyes. Typically a red dye is added to the heavier phase herein, presently the glycerin. The addition of the dye is accomplished by placing the dye in a solution of propylene glycol and adding it to the heavier liquid phase. Candle-making dyes are used to color the lighter phased polyalphaolefin oils herein. Typically a blue dye is used and the addition of the dye to the oil is accomplished by placing it in solution in very small amounts of xylene and then adding it to the oil. Coloring agents normally used in lipstick formulations are also useful in the lip gloss compositions of this invention, and at the same levels. Examples of suitable agents include FDA approved organic and inorganic pigments such as titanium dioxide, D and C. red #6 barium lake, synthetic iron oxide and the like. These materials may be incorporated at levels ranging from 1 to 8% by weight of the immiscible liquid phase in which it is used or the overall weight of the lip gloss gel.

Other conventional multiphase liquid and lip gloss ingredients and additives such as Paraban antimicrobials, antioxidants, vitamins, anti-inflammatory agents such as sodium hylorinate, and sun screens such as esters of 4-(Dimethylamino)benzoic Acid and oxybenzone may be used within the purview of the present invneiton.

The instant multiphase liquid compositions and the lip gloss can be prepared by mixing the ingredients herein in well known methods in the prior art. In one example, a light white oil designated Risella 911, manufactured by Shell Oil Co. can be used as the hydrophobic liquid phase of lighter specific gravity and isopropyl myristate as the polar organic phase of a dual phase immiscible liquid system. Contrasting coloring dyes can be added to the organic polar ester and to the Risella 911 oil to enhance the visual effect and observation of the flow of the liquid of one specific gravity with respect to the liquid of the second specific gravity. In the case of the ester and the Risella 911 oil the specific gravities are 1.26 and 0.87 respectively.

What has been described is a multiphased immiscible system primarily used in a display device for producing visual effects suitable for use as a toy and novelty device. The invention utilizes at least a two phase liquid system described above. The details set forth above and other aspects of the invention are by way of example of the scope of the invention as defined in the following claims.

What is claimed:

1. In a display device which depends on the movement of a plurality of mutually immiscible liquids, a composition comprising a mixture of at least two mutually immiscible liquids, one of which is composed primarily of a hydrophobic phase consisting essentially of liquids selected from the group of aliphatic hydrocarbons, silicone oils, polyalphaolefin oils, and mixtures thereof and the second of which comprises a polar organic phase consisting essentially of aliphatic carboxylic (fatty) acid esters the alcohol portion of the ester containing an alkyl group having carbon content of from C₇ to C₂₀.

2. The device of claim 1 wherein the immiscible liquid mixture composition comprises:
(A) at least one hydrophobic liquid substance selected from the group aliphatic alkane hydrocarbons having up to 20 carbon atoms; and
(B) at least one polar organic substance selected from the group consisting essentially of organic carboxylate esters having from 3 to 30 aliphatic carbon atoms.

3. The device of claim 2 wherein the alkane hydrocarbons are selected from the group consisting essentially of isododecane, isohexadecane, and isoeicosane.

4. The device of claim 2 wherein the polar organic esters are selected from the group consisting essentially of laureates, myristates, palmitates, stearates, oleates, and mixtures thereof.

5. The device of claim 4 wherein the polar organic esters are alkyl palmitates and/or myristates.

6. The device of claim 5 wherein the polar organic esters are isopropyl myristate and or isopropyl palmitate.

7. The device of claim 1 wherein the hydrophobic phase of the immiscible liquid composition is a silicone oil.

8. The device of claim 5 wherein the silicone oil is selected from the group consisting essentially of dimethicone and cyclomethicone.

9. The device of claim 1 wherein the hydrophobic phase of the immiscible liquid composition is a polyalphaolefin.

10. The device of claim 7 wherein the polyalphaolefin is polybutene.

11. A cosmetic composition comprising
(a) a gel comprising a low molecular weight alphaolefin copolymer and glycerin; and
(b) a mixture of at least two mutually immiscible liquids distributed throughout the gel, one of which liquids is composed primarily of a hydrophobic phase consisting essentially of liquids selected from the group of aliphatic alkane hydrocarbons, silicone oils, polyalphaolefin oils, and mixtures thereof and the second of which liquids comprises a polar organic phase consisting essentially of aliphatic carboxylic (fatty) acid esters the alcohol portion of the ester containing an alkyl group having carbon content of from C₇ to C₂₀.

3. The cosmetic composition of claim 11 wherein the alkane hydrocarbons are selected from the group consisting essentially of isododecane, isohexadecane, and isoeicosane.

13. The cosmetic composition of claim 11 wherein the polar organic esters are alkyl palmitates and/or myristates.

14. The cosmetic composition of claim 11 wherein the polar organic esters are isopropyl myristate and or isopropyl palmitate.

15. The cosmetic composition of claim 11 wherein the silicone oil is selected from the group consisting essentially of dimethicone and cyclomethicone.

16. A process for using a multiphased immiscible liquid mixture comprising:
(a) providing a composition comprising a mixture of at least two mutually immiscible liquids, one of which is composed primarily of a hydrophobic phase consisting essentially of liquids selected from the group of aliphatic hydrocarbons, silicone oils, polyalphaolefin oils, and mixtures thereof and the second of which comprises a polar organic phase consisting essentially of aliphatic carboxylic (fatty) acid esters the acid portion of the ester containing an alkyl group having carbon content of from C₇ to C₂₀.
(b) adding the mixture to a translucent container; and
(c) subjecting the container and the two phased system to energy whereby the two phases intermix forming globules and beads of the two phases resulting in a visual “lava flow” effect.

17. The process of claim 16 wherein the alkane hydrocarbons are selected from the group consisting essentially of isododecane, isohexadecane, and isoeicosane.

18. The process of claim 16 wherein the polar organic esters are alkyl palmitates and/or myristates.

19. The process of claim 18 wherein the polar organic esters are isopropyl myristate and or isopropyl palmitate.

20. The process of claim 16 wherein the silicone oil is selected from the group consisting essentially of dimethicone and cyclomethicone.
